(19) United States
(12) Patent Application Publication
(10) Pub. No.: US 2006/0280787 A1
(43) Pub. Date: Dec. 14, 2006

(54) Pharmaceutical formulation of the tubulin inhibitor indibulin for oral administration with improved pharmacokinetic properties, and process for the manufacture thereof

(75) Inventors: Berthold Roessler, Halle/Westfalen (DE); Gerhard Raab, Ronneburg (DE); Thomas Reissmann, Frankfurt am Main (DE)

Correspondence Address: PILLSBURY WINTHROP SHAW PITTMAN, LLP, P.O. BOX 10500, MCLEAN, VA, 22102, US

(73) Assignees: BAXTER INTERNATIONAL INC., Deerfield, IL (US); Baxter Healthcare S. A., Wallisellen (CH)

(21) Appl. No.: 11/151,459

(22) Filed: Jun. 14, 2005

(51) Int. Cl. A61K 9/48; A61K 9/20

(52) U.S. Cl. 424/451; 424/464

(57) ABSTRACT

The present invention relates to a pharmaceutical formulation for oral administration of the poorly soluble and therefore hardly bioavailable microtubule polymerization inhibitor Indibulin and a process for its manufacture. In particular, there is provided a pharmaceutical formulation of Indibulin for oral administration comprising a granulate containing micronized Indibulin having a particle size of less than 20 μm for at least 99% of the volume of particles, at least one hydrophilic surfactant, and at least one capsulation excipient. The present invention also discloses a method of treating hyperproliferative disorders, malignancies and neoplasms with Indibulin.

### FIELD OF THE INVENTION

The present invention relates to a specific pharmaceutical formulation for oral administration of the poorly soluble and therefore hardly bioavailable tubulin inhibitor Indibulin and a process for its manufacture.

### BACKGROUND OF THE INVENTION

During mitosis, a cell's DNA is replicated and then divided into two new cells. The process of separating the newly replicated chromosomes into the two forming cells involves spindle fibers constructed with microtubules, which themselves are formed by long chains of smaller protein subunits called tubulins. Spindle microtubules attach to replicated chromosomes and pull one copy to each side of the dividing cell. Without these microtubules, cell division is not possible.

Microtubules therefore are among the most important sub-cellular targets of anticancer chemotherapeutics because they are present in all cells and are necessary for mitotic, interphase and cell maintenance functions (e.g., intracellular transport, development and maintenance of cell shape, cell motility, and possibly distribution of molecules on cell membranes). Compounds that interact with tubulin can interfere with the cell cycle by causing tubulin precipitation and sequestration, thereby interrupting many important biologic functions that depend on the microtubular class of subcellular organelles. Therefore, such compounds can potentially inhibit the proliferation of tumor cell lines derived from various organs. See, e.g., Bacher et al. (2001) Pure Appl. Chem. 73:9 1459-1464 and Rowinsky & Donehower (1991) Pharmac. Ther. 52:35-84.

Accordingly, new, synthetic, small-molecule chemical entities that bind to tubulin, but are neither a substrate of transmembrane pumps nor interfere with the function of axonal microtubules, would strongly increase the therapeutic index in the treatment of malignancies.

A series of synthetic molecules that bind to tubulin are currently being evaluated in the preclinical or early clinical stage. Among them is the synthetic compound, N-(pyridine-4-yl)-[1-(4-chlorobenzyl)-indole-3-yl]glyoxylic acid amide, named Indibulin (INN) having the formula C₂₂H₁₆CIN₃O₂ and the following structure:

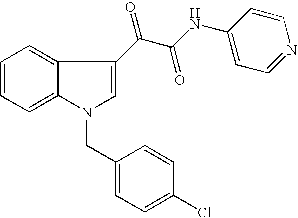

Indibulin is a synthetic small molecule tubulin inhibitor with significant antitumor activity in vitro and in vivo. It inhibits polymerization of microtubules in tumor cells, as well as in a cell-free system. The binding site of Indibulin does not appear to overlap with the tubulin-binding sites of the well-characterized microtubule-destabilizing agents vincristine or colchicine. Furthermore, the molecule selectively blocks cell cycle progression at metaphase.

In vitro, Indibulin exerts significant antitumor activity against a variety of malignancies (e.g., prostate, brain, breast, pancreas, and colon). Indibulin displays high in vivo anti-neoplastic efficacy in animals. Based on its mechanism of action, it is expected to target all types of solid tumors. It is also expected to exhibit anti-asthmatic, anti-allergic, immunosuppressant and immunomodulating actions. No neurological symptoms have so far been found in animal experiments. In preclinical experiments in rodents, the compound was very well tolerated at highly effective doses. Another advantage for further development is its easy synthesis, in contrast to other tubulin-inhibitory compounds.

Indibulin is obtained by chemical synthesis as a white crystalline powder. Its solubility in hydrophilic solvents is poor. For example, it is practically insoluble in water, methanol, ethanol or 2-propanol. Due to these properties, the bioavailability of pure Indibulin is very low, as is Indibulin's bioavailability in common pharmaceutical dosage forms, e.g., powder, granula, tablets or capsules.

In various organic solvents, e.g., dimethylformamide, dimethylsulfoxide and N-methylpyrrolidone, Indibulin shows sufficient solubility. However, these organic solvents cannot be used for application in humans due to their toxicity.

In addition, highly concentrated (roughly >50% w/v) organic acids, e.g., acetic acid or lactic acid, are relatively good solvents for Indibulin.

Various technologies are known, proven and applicable for the improvement of the bioavailability of poorly soluble drugs.
(i) The process of micronization, wherein the active ingredient and formulation are reduced to ultrafine size (1 to 10 microns), for use in oral dosage forms, for example, suspensions, capsules or tablets. See R. Voigt, Lehrbuch der Pharm. Tech.; Hagers Handbuch Band 2, Kap. 12.2; Bauer, Frömming, Führer, Pharmazeutische Technologie. However, in the case of Indibulin, these types of formulation lead to a relatively low and insufficient bioavailability and therefore to low plasma levels and no efficacy.
(ii) The dissolution or suspension of active ingredients into organic solvents and surfactants. See R. Voigt, Lehrbuch der Pharm. Tech; Hagers Handbuch Band 2, Kap. 12.2; Bauer, Frömming, Führer, Pharmazeutische Technologie. However, the use of surfactants leads to an increased bioavailability of Indibulin in animal tests, but in all cases the formulation were not acceptable for human use, due to the high amount of excipients needed.
(iii) The preparation of colloidal suspensions, nano- or microparticle suspensions. For example, by using high shear forces the substance is crushed to nanoparticulate size or the substance is dissolved and afterwards precipitated out of a solvent mixture. For stabilization, often surfactants and/or salts are added. Additionally the viscosity of the suspension can be modified to decrease sedimentation. See also U.S. Pat. No. 4,826,689. However, the manufacturing and processing of this type of pharmaceutical formulation is of extremely high complexicity.
(iv) The preparation of a drinking solution of active ingredients. For Indibulin, it is additionally known that an oversaturated solution in lactic acid can be prepared and orally administered (see also DE 2004 031538.8). Due to stability reasons, this solution has to be freshly prepared prior to administration. Such a solution shows good bioavailability of Indibulin, but due to the relatively high concentration of lactic acid (5 to 10% w/v), the amount which can be administered is limited by taste and side effects. Since the concentration of the ready to use solution is approximately 1 mg/ml in 10% lactic acid, the applicable volume is limited to roughly 60 to 80 ml.

Therefore, a strong need exists for a new pharmaceutical Indibulin formulation which exhibits improved bioavailability of Indibulin without showing the disadvantages given in the prior art as mentioned above. Thus, it is an object of the present invention to provide a new pharmaceutical formulation exhibiting improved bioavailability of the pure Indibulin substance. It is a further object of the present invention to provide a respective method for the manufacture of such a pharmaceutical formulation.

Other objects, features, and advantages of the present invention will be apparent to those skilled in the art from a consideration of the following detailed description of preferred exemplary embodiments, thereof.

### SUMMARY OF THE INVENTION

The present invention relates to an improved pharmaceutical formulation of Indibulin for oral administration comprising a granulate containing micronized Indibulin having a particle size of less than 20 μm for at least 99% of the volume of particles, at least one hydrophilic surfactant, and at least one additional capsulation excipients. Further, the present invention relates to a tablet prepared by using said pharmaceutical formulation and a capsule filled with said pharmaceutical formulation, respectively.

According to the present invention, the pharmaceutical formulation of Indibulin is based on micronization of Indibulin combined with a granulation procedure using a hydrophilic surfactant (e.g., polysorbate, poloxamer, cremophor) and at least one common capsulation excipients (e.g., cellulose, starch, highly disperse silicon dioxide, etc). This leads to a sufficient bioavailability and therefore effective plasma levels, which is a significant improvement in formulation of the poorly soluble drug Indibulin. Compared with an ordinary capsule or tablet made of micronized Indibulin, the bioavailability of these novel pharmaceutical formulations of Indibulin is significantly higher according to the present invention. The pharmaceutical formulations of Indibulin of the present invention are on the same level as found for the drinking solution of lactic acid, but avoid the disadvantages of being limited to the low dosage for the lactic acid solution.

Additionally, a process for manufacturing said pharmaceutical formulation, comprising the steps of micronizing Indibulin to a particle size of less than 20 μm for more than 99% of the volume of particles and homogeneously mixing the micronized Indibulin with at least one hydrophilic surfactant and additional capsulation excipients, is provided according to the present invention.

The present invention also provides for a method of treating a variety of hyperproliferative disorders, malignancies and neoplasms (specifically solid tumors) with Indibulin, including but not limited to, cancers of the abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Similarly, other hyperproliferative disorders can also be treated by the method of the present invention include, but are not limited to, hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

### DETAILED DESCRIPTION OF FIGURES

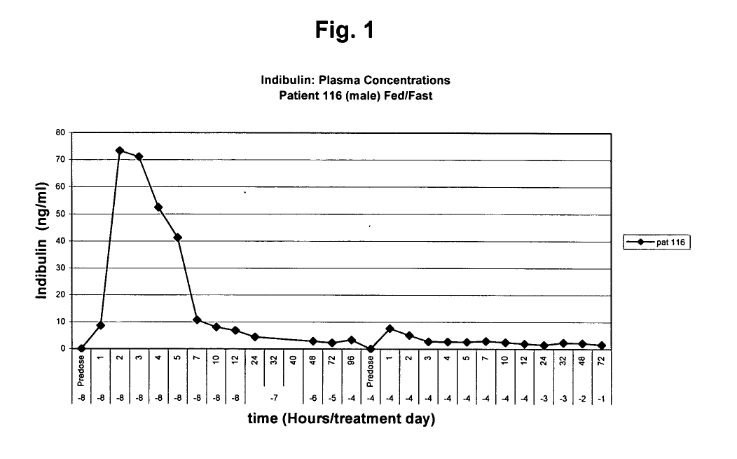

FIG. 1 shows the result of a bioavailability study in humans by treating with a formulation according to the present invention first under fasted and for second treatment under fed conditions afterwards.

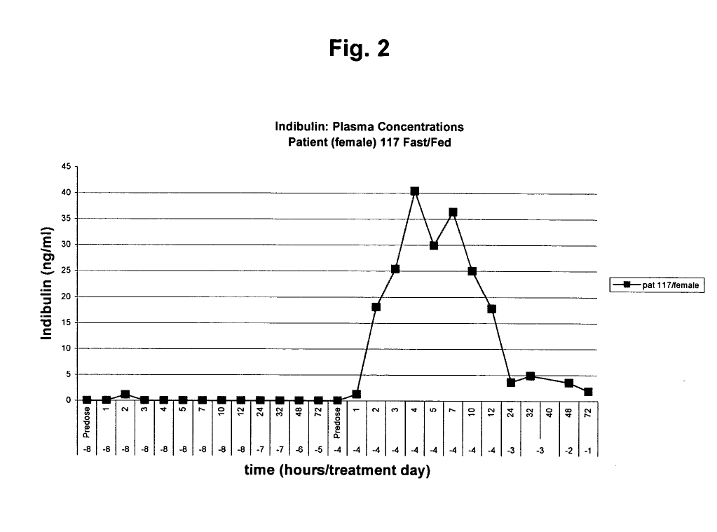

FIG. 2 shows data of said bioavailability study from a patient who was first treated fed and afterwards treated under fasted conditions.

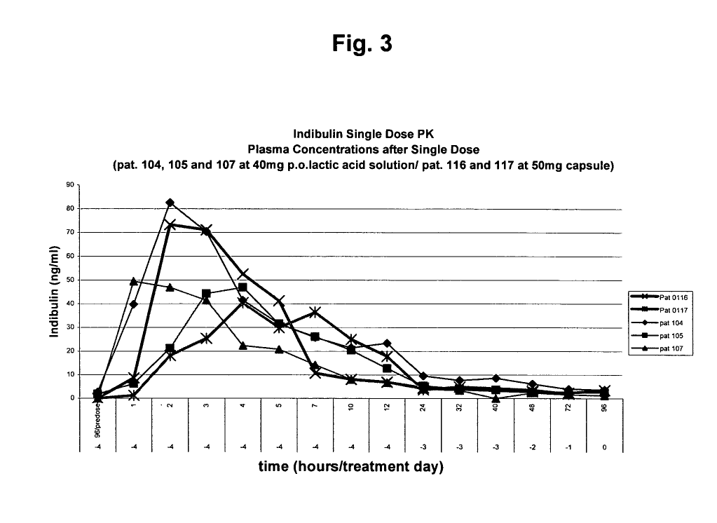

FIG. 3 shows the plasma level from 5 patients treated either with the pharmaceutical formulation according to the present invention as obtained in Example 1 herein below or the drinking solution, Example 4, for comparison.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated.

One aspect of the present invention relates to a pharmaceutical formulation of Indibulin for oral administration comprising a granulate containing micronized Indibulin having a particle size of less than 20 μm for at least 99% of the volume of particles, at least one hydrophilic surfactant, and at least one capsulation excipient. Preferably, the micronized Indibulin has a particle size of less than 10 μm for at least 90% of the volume of particles. More preferably, the micronized Indibulin has a particle size of less than 10 μm for at least 99% of the volume of particles. Even more preferred, the micronized Indibulin has a mean particle size in the range of 2 to 4 μm.

In a preferred embodiment of the present invention, the pharmaceutical formulation comprises (1) Indibulin in an amount of about 10 to about 50 percent weight/volume, (2) at least one hydrophilic surfactant in an amount of about 1 to about 10 percent weight/volume, and (3) at least one capsulation excipient in an amount of about 40 to about 80 percent weight/volume, wherein the three constituents always add up to 100 percent weight/volume of said pharmaceutical formulation.

The hydrophilic surfactant is not subject to any particular limitation as long as it is capable of acting as an oil-in-water surfactant. Preferably, the hydrophilic surfactant(s) is/are selected from the group consisting of polysorbates, poloxamers, cremophors and polyalkylene glycols. Any type of polysorbate can be employed, but particularly the polysorbate is selected from polysorbate 20, polysorbate 40, polysorbate 60 or polysorbate 80, more preferred from polysorbate 80. Further, any type of poloxamers can also be employed. Poloxamers are surfactant-like block polymers having a central polypropylene glycol moiety with a macrogol moiety connected on both terminal ends. Typical poloxamers suited for the present invention are poloxamers 188 and 407, particularly poloxamer 188. Cremaphors can also be used as a hydrophilic surfactant. Cremophors are non-ionic emulsifiers obtained by causing ethylene oxide to react with castor oil particularly in a molar ratio of about 35 moles to 1 mole. Other common names are polyoxyethyleneglycerol triricinoleate 35 or polyoxyl 35 castor oil. A typical cremophor is, for example, Cremophor® EL supplied by BASF AG, Germany.

Capsulation excipients are a component of the present invention's pharmaceutical formulation. Capsulation excipients are those which are common in the art and can be suitably used in the present invention. In particular, those capsulation excipients can comprise cellulose such as microcrystalline cellulose or a derivative thereof, gelatine, starch, particularly corn starch, and highly disperse silicon dioxide (aerosil). Typically, the capsulation excipients comprise a mixture of microcrystalline cellulose, gelatine, corn starch and aerosil. For example, corn starch and microcrystalline cellulose can serve as a filling mass and degradants. Highly disperse silicon dioxide (aerosil) acts in turn to make the mass fluent. Gelatine usually serves as an adhesive to get homogeneous granules.

In a preferred embodiment of the present invention, the granules constituting said pharmaceutical formulation are covered by an outer phase composed of a mixture comprising starch, particularly corn starch, highly dispersed silicon dioxide and magnesium stearate. Such an outer phase properly enables the encapsulation the granules.

A further aspect of the present invention relates to a tablet prepared by using the pharmaceutical formulation according to the present invention. Another aspect of the present invention relates to a capsule filled with said pharmaceutical formulation. Thus, the pharmaceutical formulation according to the present invention can be suitably used as a capsule filling mass. Such a capsule can particularly be a hard gelatine capsule of size 1 or 2 (Ph. Eur.).

In such a capsule according to the present invention, the amount of Indibulin as pharmaceutically active ingredient is preferably in the range of about 20 to about 100 mg, more preferably about 30 to about 70 mg, even more preferably about 50 mg per capsule.

A further aspect of the present invention relates to a process for manufacturing said pharmaceutical formulation, comprising the steps of micronizing Indibulin to a particle size of less than 20 μm for more than 99% volume of the particles and homogeneously mixing the micronized Indibulin with at least one hydrophilic surfactant and one or more capsulation excipients. Because Indibulin is practically insoluble in water, micronization can enhance the dissolution rate of drugs which are not readily bioavailable. Jet milling is one method of micronization. Jet milling pulverizes larger sized particles into smaller sized particles by using compressed air to propel the larger sized particles into each other to create the smaller sized particles. The smaller particles exit while the larger particles remain in the milling chamber. Because size reduction is dependent on collisions between particles, jet mills can reduce the risk of contamination and/or attritional heat. Preferably, the Indibulin is micronized by milling with a jet mill.

In a preferred embodiment of the present invention, the micronized Indibulin is homogeneously mixed with corn starch, microcrystalline cellulose and aerosil to obtain a powder mixture, while simultaneously gelatine and polysorbate are dissolved in purified water, and subsequently the powder mixture is moistened with the gelatine-polysorbate solution to obtain a homogeneous granulate by sieving through 0.8 mm sieve.

The process according to the present invention can further comprise the step of encapsulating the granules by mixing with an outer phase forming mixture which in turn is obtained by mixing corn starch, aerosil and magnesium stearate.

Moreover, the process according to the present invention can further comprise the step of filling the pharmaceutical formulation in hard gelatine capsules of size 1 or 2 (Ph. Eur.) or, alternatively, the pharmaceutical formulation is subsequently processed for tabletting.

Because Indibulin falls within a class of molecules that can inhibit microtubule polymerization, Indibulin may be useful in the treatment of a number of hyperproliferative disorders, malignancies and neoplasms, including solid tumors. The present invention also provides for a method of treating such hyperproliferative disorders, malignancies and neoplasms. For their use in treating hyperproliferative disorders, malignancies and neoplasms, Indibulin may be present as part of pharmacologically active compositions suitable for the treatment of animals, particularly humans. The microtubule polymerization inhibitor (i.e., Indibulin) containing composition must come into contact with microtubules, wherein microtubules are then destabilized in hyperproliferative cells and/or tumor cells.

The preferred dosage of Indubulin for the treatment of hyperproliferative disorders, malignancies and neoplasms will vary depending upon the hyperproliferative disorders, malignancies and neoplasms in question and the patient's weight and age. The number of administrations of Indibulin will also vary according to the response of the individual patient to the treatment. For the treatment of hyperproliferative disorders, such as cancer, suitable dosages of the microtubule polymerization inhibitor occur in amounts between 0.5 mg/kg of body weight to 100 mg/kg of body weight per day, preferably of between 1.0 mg/kg of body weight to about 20 mg/kg of body weight. Moreover, tubulin inhibition assays can also provide one of skill in the art with the appropriate concentrations of Indibulin necessary to hyperproliferative cells, and the appropriate dosage can be calculated from that information.

### EXAMPLES

The invention is described in the following examples in more detail, but without being limited to those.

### Example 1

### Capsule Formulation with a Strength of 50 mg Indibulin

In order to increase the specific surface of the drug substance Indibulin, it is milled via a jet mill. The resulting particle size should be less than 10 μm for more than 90% (volume) of the particles with an average size of about 2 to 4 μm.

The micronized Indibulin is homogeneously mixed with corn starch, microcrystalline cellulose and Aerosil. In parallel, gelatine and polysorbate is dissolved in purified water. The powder mixture is then moistened with the gelatine-polysorbate-solution in order to get a homogeneous granulate by sieving through 0.8 mm sieve.

To enable encapsulation, the granula is mixed with an outer phase of the capsule mass which is obtained by mixing corn starch, Aerosil and magnesium stearate.

The completed capsule filling mass is then filled in hard gelatine capsules of size 2 (Ph. Eur.)

Composition per unit (Capsule)

Granulate

| | | |
|---|---|---|
| Indibulin | 50.0 | mg |
| corn starch | 40.0 | mg |
| aerosil | 3.0 | mg |
| gelatine | 2.5 | mg |
| polysorbate 80 | 5.0 | mg |
| microcrystalline cellulose | 45.0 | mg |

| | |
|---|---|
| purified water (USP, EP) | q.s. |

Outer phase

| | | |
|---|---|---|
| corn starch | 10.0 | mg |
| aerosil | 2.5 | mg |
| Mg stearate | 2.0 | mg |
| hard gelatine capsules of size 2 | 1 | |

### Example 2

### Capsule Formulation with a Strength of 100 mg Indibulin

The manufacturing of a 100 mg strength of Indibulin capsules follows the description in Example 1, but having a slightly different composition per unit.

Composition per unit (Capsule)

Granulate

| | | |
|---|---|---|
| Indibulin | 100.0 | mg |
| corn starch | 80.0 | mg |
| aerosil | 6.0 | mg |
| gelatine | 5.0 | mg |
| polysorbate 80 | 10.0 | mg |
| microcrystalline cellulose | 90.0 | mg |

| | |
|---|---|
| purified water (USP, EP) | q.s. |

Outer phase

| | | |
|---|---|---|
| corn starch | 20.0 | mg |
| aerosil | 5.0 | mg |
| Mg stearate | 4.0 | mg |
| hard gelatine capsules of size 1 | 1 | |

### Example 3

### Capsule Formulation with a Strength of 50 mg Indibulin using a poloxamer

Composition per unit (Capsule)

Granulate

| | | |
|---|---|---|
| Indibulin | 50.0 | mg |
| corn starch | 40.0 | mg |
| aerosil | 3.0 | mg |
| gelatine | 2.5 | mg |
| poloxamere 188 | 5.0 | mg |
| microcrystalline cellulose | 45.0 | mg |

| | |
|---|---|
| purified water (USP, EP) | q.s. |

Outer phase

| | | |
|---|---|---|
| corn starch | 10.0 | mg |
| aerosil | 2.5 | mg |
| Mg stearate | 2.0 | mg |
| hard gelatine capsules of size 2 | 1 | |

### Example 4

### Drinking Solution of Indibulin in 10% Lactic Acid (1 mg/ml)

For preparation of the drinking solution, a certain amount of the pure active compound is dissolved in lactic acid 90% (Ph. Eur.). Afterwards the obtained solution is diluted with an aqueous solution of glucose and passion fruit flavor to the applicable volume and concentration. The final solution is oversaturated and therefore only stable for 2 hours. Therefore the drinking solution has to be prepared directly prior to administration.

The applicable formulation contains 60 ml of an aqueous drinking solution of Indibulin with a concentration of 1 mg/ml. Glucose and passion fruit flavor are used to modify the taste to make swallowing easier.

Composition of the solution:

| | |
|---|---|
| Indibulin | 60.0 mg |
| lactic acid 90% | 7269.2 mg |
| glucose (Ph.Eur.) | 5532.5 mg |
| passion fruit flavor | 96.9 mg |
| pur. water | 50503.7 mg |

### Example 5

### Bioavailability Studies on Animal

Pharmacokinetic studies were carried out in Cynomolgus monkeys, comparing the bioavailability of Indibulin from three different formulation for oral administration and for reference from an intravenously administered solution of Indibulin in solutol®/propane diol:
1. formulation according to the present invention as obtained in Example 1 (50 mg)
2. standard capsule of micronized Indibulin (50 mg)
3. drinking solution of Indibulin in 10% lactic acid, as described in Example 4
4. intravenously administered solution of Indibulin in solutol®/propane diol.

The results show a significant improved bioavailability for the formulation according to the present invention compared with an ordinary capsule formulation. In comparison with the drinking solution containing lactic acid, the bioavailability from the formulation according to the present invention as obtained in Example 1 is lower, but this is compensated by the better tolerability and the higher possible dosing as exemplified by said Example 1; cf. Table 1 herein below (AUC=area under curve).

TABLE 1
Meanₐᵣ ± SD (n = 6)

| Admin. | | Animal | AUC₀₋₂₄* | AUC_{0-24, norm}* | AUC₀₋₃₆* | AUC_{0-36, norm}* |
|---|---|---|---|---|---|---|
| Route | Treatment | group | [ng · h/ml] | [ng · h/ml] | [ng · h/ml] | [ng · h/ml] |
| perorally | formulation according to | 1a | 524 ± 628 | 429 ± 473 | 561 ± 695 | 455 ± 510 |
| | the present invention as | | | | | |
| | obtained in Example 1 | | | | | |
| | (50 mg) | | | | | |
| perorally | standard caps (50 mg) | 1b | 76.6 ± 114 | 82.1 ± 139 | 103 ± 113 | 109 ± 137 |
| perorally | solution (10 mg/kg) | 1a | 1886 ± 1085 | 1886 ± 1085 | 2863 ± 1810 | 2863 ± 1810 |
| | in 10% lactic acid | | | | | |
| intravenously | solution (0.2 mg/kg) | 1b | 299 ± 85.4* | 14949 ± 4270* | — | — |
| | in sol/prop* | | | | | |

*Plasma samples from intravenously administered animals were only withdrawn until 4 hours and, thus, only AUC₀₋₄ could be calculated

### Example 6

### Bioavailability Studies in Humans

The formulation of Example 1 was tested in Phase I studies in humans. Patients were treated with the Indibulin capsules under fed and fasted conditions to evaluate the influence of administration prior or after a meal.

To obtain relevant plasma levels it seems to be better to administer the capsules under fed conditions. FIG. 1 shows treatment first under fed and for second treatment under fasted conditions afterwards. Good bioavailability can be observed in the first treatment whereas after second treatment no plasma level was found.

FIG. 2 shows data from a patient who was first treated fasted and afterwards treated under fed conditions. Again, if patient was fasted, no plasma level of Indibulin can be found, but under fed conditions significant plasma levels were observed.

### Example 7

### Comparison of the Bioavailability of Example 1 (Capsule Formulation According to the Present Invention) and Example 4 (Drinking Solution) in Phase I Studies in Humans

FIG. 3 shows the plasma levels of Indibulin from three patients (patients 104, 105 and 107) treated with 40 mg via the lactic acid drinking solution versus two patients (patients 116 and 117) treated with 50 mg via the capsule formulation according to the present invention.

The plasma levels of both formulation were within the same range taking the standard deviation into account, therefore no significant differences can be found. The bioavailability can be stated to be similar for both formulations.

Those skilled in the art will further appreciate that the present invention may be embodied in other specific forms without departing from the spirit or central attributes thereof. In that foregoing description of the present invention discloses only exemplary embodiments thereof, it is to be understood that other variations are contemplated as being within the scope of the present invention. Accordingly, the present invention is not limited to the particular embodiments that have been described in detail herein. Rather, references should be made to the appended claims as indicative of the scope and content of the invention.

1. A composition comprising a granulate containing micronized Indibulin having a particle size of less than 20 μm for at least 99% of the volume of particles, at least one hydrophilic surfactant, and at least one capsulation excipient.

2. The composition according to claim 1, wherein the micronized Indibulin has a particle size of less than 10 μm for at least 90% of the volume of particles.

3. The composition according to claim 1, wherein the micronized Indibulin has a particle size of less than 10 μm for at least 99% of the volume of particles.

4. The composition according to claim 1, wherein the micronized Indibulin has a mean particle size in the range of 2 to 4 μm.

5. The composition according to claim 1, comprising Indibulin in an amount of about 10 to about 50 percent weight/volume, the hydrophilic surfactant in an amount of about 1 to about 10 percent weight/volume, and the additional capsulation excipients in an amount of about 40 to about 80 percent weight/volume.

6. The composition according to claim 1, wherein the hydrophilic surfactant is selected from the group consisting of polysorbates, poloxamers, cremophors and polyalkylene glycols.

7. The composition according to claim 6, wherein the polysorbate is selected from the group consisting of polysorbate 20, polysorbate 40, polysorbate 60 and polysorbate 80.

8. The composition according to claim 6, wherein the poloxamer is selected from the group consisting of poloxamer 188 and poloxamer 407.

9. The composition according to claim 6, wherein the cremophor is Cremophor® EL.

10. The composition according to claim 1, wherein the capsulation excipient comprises at least one selected from the group consisting of microcrystalline cellulose and a derivative thereof, gelatine, starch, and highly disperse silicon dioxide.

11. The composition according to claim 10, wherein the starch is corn starch.

12. The composition according to claim 1, wherein the granulate constituting said composition are covered by an outer phase composed of a mixture comprising starch, highly dispersed silicon dioxide and magnesium stearate.

13. The composition according to claim 12, wherein the starch is corn starch.

14. A tablet prepared by using the composition as defined in claim 1.

15. A capsule filled with a composition comprising a granulate containing micronized Indibulin having a particle size of less than 20 μm for at least 99% of the volume of particles, at least one hydrophilic surfactant, and at least one capsulation excipient.

16. The capsule according to claim 15 which is a hard gelatine capsule of size 1 or 2.

17. A capsule according to claim 16 wherein the amount of Indibulin as pharmaceutically active ingredient is in the range of about 20 to about 100 mg, preferably about 30 to about 70 mg, more preferably about 50 mg per capsule.

18. A process for manufacturing a composition comprising a granulate containing micronized Indibulin having a particle size of less than 20 μm for at least 99% of the volume of particles, at least one hydrophilic surfactant, and at least one capsulation excipient, having the steps of:
(a) micronizing Indibulin to a particle size of less than 20 μm for more than 99% of the volume of particles; and
(b) homogenizing the micronized Indibulin with at least one hydrophilic surfactant and at least one capsulation excipient.

19. The process according to claim 18, wherein the Indibulin is micronized by milling with a jet mill.

20. The process according to claim 18, wherein the micronized Indibulin is homogeneously mixed with corn starch, microcrystalline cellulose and aerosil to obtain a powder mixture, while simultaneously gelatine and polysorbate are dissolved in purified water, and subsequently the powder mixture is moistened with the gelatine-polysorbate solution to obtain homogeneous granules by sieving through 0.8 mm sieve.

21. The process according to claim 18, further comprising the step of encapsulating the granules by mixing with an outer phase forming mixture which in turn is obtained by mixing corn starch, aerosil and magnesium stearate.

22. The process according to claim 18, further comprising the step of filling the composition in hard gelatine capsules of size 1 or 2.

23. The process according to claim 18, wherein the composition is processed for tabletting.
